(19) United States
(12) Patent Application Publication
(10) Pub. No.: US 2005/0192482 A1
(43) Pub. Date: Sep. 1, 2005

(54) Disposable sheath for specula

(75) Inventors: Janette M. Carpenter, Tucson, AZ (US); Marvin J. Slepian, Tucson, AZ (US)

Correspondence Address: PATREA L. PABST;PABST PATENT GROUP LLP, 400 COLONY SQUARE, SUITE 1200, ATLANTA, GA, 30361, US

(73) Assignee: Endoluminal Therapeutics, Inc.

(21) Appl. No.: 11/046,611

(22) Filed: Jan. 28, 2005

Related U.S. Application Data
(60) Provisional application No. 60/540,596, filed on Jan. 30, 2004.

(51) Int. Cl. A61B 1/32

(52) U.S. Cl. 600/203

(57) ABSTRACT

A speculum sheath which covers a speculum and methods for using the sheath are described herein. A variety of different types of specula may be covered with the sheath, including anal, vaginal, ear, and nasal specula. The sheath is made of a compliant or partially compliant material, including latex, vinyl, natural and synthetic rubbers, silicone, nylon, polyethylene, polypropylene, and non-degradable or degradable elastomers. The material may be transparent or opaque. In the preferred embodiment, the material is transparent. The sheath may contain one or two openings. Optionally, the sheath contains an affixing means on the inside of or at least one of the openings on the sheath to prevent slippage when in contact with the speculum. Optionally, the sheath contains an active agent, such as hemostatic agents, anti-infectives, antibiotics, antimitotics anti-inflammatory, or other chemotherapeutic agents. In the preferred embodiment, the sheath is placed on a vaginal speculum and supports loose vaginal tissue when inserted into a patient. In the most preferred embodiment, the material is transparent so that medical provider may view the lumen.

### CROSS-REFERENCE TO RELATED APPLICATIONS

This application claims priority to U.S. Ser. No. 60/540,596, entitled “Disposable Sheath for Specula” to Janette M. Carpenter and Marvin J. Slepian, filed Jan. 30, 2004.

### FIELD OF THE INVENTION

The present invention relates to a sheath for a speculum and methods for improving the examination and treatment of patients using specula.

### BACKGROUND OF THE INVENTION

Many types of medical specula for a physician's use in dialating an aperture in a patient's body in a medical examination are known and commercially available. Examples include nasal specula, ear specula, anal specula, and vaginal specula. Examples of some designs for manually operated nasal specula are disclosed in U.S. Pat. No. 4,576,168 to Jalowayski and U.S. Pat. No. 5,772,582 to Huttner et al. Examples of ear specula are described in U.S. Pat. No. 5,293,862 to O'Hara et al., U.S. Pat. No. 6,155,987 to Scherl and U.S. Pat. No. 4,441,485 to Reynolds. The ear and nasal specula are used during patient examination or surgery for a variety of procedures, including submucous resection of the nasal septum (SMR), septorhinoplasty, microscopic sinus or otic surgery, polypectomy, myringtomy and grommet insertion, and stapedectomy.

A vaginal speculum is a medical device having a pair of protruding blades that are inserted into the vagina and can then be opened in a parallel and/or angular manner. Two categories of vaginal specula currently exist, specula made from metal that can be sterilized, and specula made from plastic that are disposable. Examples of the vaginal speculum are found in U.S. Pat. No. 3,815,585 to Fiore; U.S. Pat. No. 3,890,961 to Moore et al.; and U.S. Pat. No. 4,686,966 to Tsai.

U.S. Pat. No. 4,492,220 to Hayes describes a pair of disposable covers, one for each blade of the vaginal speculum. The primary object of the disposable covers is to minimize the possibility of cross-contamination between patients when the same non-disposable speculum is used for many patients. Because there are two separate blade covers, one for each blade, the covers do not assist in the clear visualization of the vaginal tissues or of the cervix.

U.S. Pat. No. 5,007,409 to Pope relates to the use of an insulating sheath to cover speculum blades with the intent of reducing pain and the shock of cold metal speculum blades during gynecological examinations. These blade covers are described as silicone rubber sheaths molded to substantially conform to both the interior and exterior surfaces of each blade of a speculum. Again, these blade cover sheaths, like the aforementioned vaginal speculum protector, do not cover the entire speculum and do nothing to better the view of the vagina and cervix.

U.S. Pat. No. 5,823,191 to Cho and U.S. Pat. No. 5,579,784 to Harari describe condoms used for contraceptive purposes. They consist of a condom body and a small sack for collecting semen. The condoms are made of latex and unrolled to cover the penis from its head to the pubic base. Because the condom is closed at one end it cannot be used as a vaginal speculum sheath. In order to be used as a vaginal speculum sheath for the purpose of restraining loose vaginal tissue and then being able to perform testing or treatment of a cervix, both ends of the sheath need to be open.

When a medical provider performs a gynecological pelvic exam on a woman who is overweight, large or who has given birth to many children, the vaginal tissue is often very loose, lax or redundant. When a medical provider inserts a speculum into the vagina and attempts to visualize the cervix, this loose vaginal tissue falls into the vaginal cavity through the sides of the open, duckbill speculum, thereby blocking the view of the cervix. It is important for a medical provider to be able to visualize a woman's cervix or other structures in the vaginal vault for numerous reasons, including: evaluating the health of the cervix by visualization, obtaining thorough pap smear samples of both the endo and ecto cervix, obtaining biopsies of abnormal vaginal or cervical tissues removing abnormal vaginal or cervical growths, and checking pregnant women to be sure that the cervical os (opening) is closed during pregnancy. If the vaginal tissue falls into the vagina and blocks the view of the cervix, the previous assessments, treatments, diagnostic and therapeutic procedures cannot be performed thoroughly. Further, any biopsies, surgeries or other therapies affected may be compromised by bleeding or tissue interference, if proper maintenance of structures in their place cannot be carried out.

Therefore it is an object of the invention to provide a device that allows a medical provider to support tissue in a lumen when a speculum is used.

It is a further object of the invention to provide a method for improving the examination and treatment of an area in a patient while using a speculum.

### BRIEF SUMMARY OF THE INVENTION

A speculum sheath which covers a speculum and methods for using the sheath are described herein. A variety of different types of specula may be covered with the sheath, including anal, vaginal, ear, and nasal specula. The sheath is made of a compliant or partially compliant material, including latex, vinyl, rubber, nylon, polyethylene, and polypropylene. The material may be transparent or opaque. In the preferred embodiment, the material is transparent. The sheath may contain one or two openings. Optionally, the sheath contains an affixing means on the inside of the sheath to prevent slippage when in contact with the speculum. In another embodiment, the sheath may have adhesive and/or a hemostatic agent on the outer surface. In another embodiment the sheath may have other releasable therapeutic medications, such as antiinfectives, antibiotics, antimitotics anti-inflammatory, or other chemotherapeutic agents. In the preferred embodiment, the sheath is placed on a vaginal speculum and supports loose vaginal tissue when inserted into a patient. In the most preferred embodiment, the material is transparent so that medical provider may view the lumen.

### BRIEF DESCRIPTION OF THE DRAWINGS

FIG. 1A is a full length view of a vaginal speculum sheath.

FIG. 1B is a top view of a vaginal speculum sheath-rolled up.

FIG. 1C is a side view of a vaginal speculum sheath-rolled up.

FIG. 1D is a full length view, showing both open ends of a vaginal speculum sheath.

FIG. 2A is a side view of a vaginal speculum sheath on a vaginal speculum.

FIG. 2B is a front view of a vaginal speculum sheath on a vaginal speculum.

### DETAILED DESCRIPTION OF THE INVENTION

Sheaths for placement on specula and methods for using such sheaths are described herein.

I. Devices

A device containing a sheath and a speculum may be inserted into a lumen in a patient.

A. Sheath

In the preferred embodiment, the sheath is formed from one continuous material. Optionally, the sheath is composed of a multiplicity of materials which are aligned to form a continuum. The sheath contains at least one opening. In the preferred embodiment, the sheath contains two openings. The material may be transparent or opaque. Optionally, at least one end contains a ring of material that is thicker than the material used in the remainder of the sheath. Alternatively, two openings on the sheath may contain a ring of thicker material. The material used at the opening may be the same as the material used for the rest sheath or may be different than the material used for the rest of the sheath. In the preferred embodiment, the sheath is designed to be affixed to the speculum in a manner that prevents slippage of the sheath. For example, in one embodiment, at least one of the openings is configured to hook under or over the lips of the vaginal speculum. In another embodiment, at least one of the openings is formed of a material that is able to adhere to the speculum to prevent slippage of the sheath.

1. Materials

The sheath is formed from a compliant or partially compliant material. Materials may be directionally compliant, i.e. anisotropic, to allow preferential expansion in one plane. For example, the material may allow for expansion in the lateral plane or in the anterior-posterior plane. Suitable materials include latex, vinyl, natural and synthetic rubbers, silicone, nylon, polyethylene, and polypropylene and other non-degradable or degradable elastomers.

Different material properties provide support in different directions. If a material is stiff, then it may open only partially on the speculum, while providing a relatively stiff sidewall, hence good tissue support. Alternatively, if a material is compliant, it may stretch readily, allowing the speculum to open as wide as necessary, but providing only partial support to the surrounding tissue. By varying the properties of the material used to form the sheath, a sheath which is sufficiently pliable and strong may be selected, to allow for opening the speculum and supporting the surrounding tissue.

For example, the sheath may be fabricated from silicone rubber. The continuous cylindrical sheath may be fashioned so that two opposing arcs of the sheath's circular cross-sectional profile may be thicker and hence stiffer while the remaining two arcs are thinner and compliant (e.g. if one was looking at the face of a clock, the material would be thick from 12 to 3 and 6 to 9 on the clock face, and thin at 3 to 6 and 9 to 12). In another embodiment, the material may be pre-stretched or otherwise mechanically, chemically, electrically irradiated, or otherwise treated to selectively change the material properties in defined zones of the sheath to achieve varying mechanical properties at differing locations. In a third example, materials of differing material properties may be fabricated together creating a continuum with regions of differing properties.

2. Size and Shape

For vaginal specula, the sheath is cylindrical. The length ranges from 5 cm to 15 cm and the diameter of the openings range from 1.0 cm to 20 cm. The thickness of the material typically ranges from 0.5-5 mm. For the thick ring on the sheath, the thickness is about 2 times thicker than the remainder of the sheath, generally ranging from 1.0-3.0 mm.

For nasal specula, the sheath is cylindrical or has a modified cylindrical shape, including a curved “banana-like” shape, trumpet-like, or tubular with regions of asymmetric outpouching to accommodate underlying tissue terrains. The length of the sheath generally ranges from 1 mm to 35 mm. The opening has a diameter of 2 mm to 120 mm. The length of the speculum ranges from 1 cm to 12 cm.

For anal specula, the sheath may be cylindrical or a modified cylindrical shape, including a curved “banana-like” shape, trumpet-like, or tubular with regions of asymmetric outpouching to accommodate underlying tissue terrains. The opening has a diameter of 5 mm to 150 mm. The length of the speculum ranges from 1 cm to 25 cm.

3. Affixing Means

Optionally, the sheath contains affixing means on the inside or at least one of the ends of the sheath to prevent slippage when in contact with the speculum. Suitable affixing means include an adhesive, a bonding material, ribs, hooks, and buttons. The sheath may be glued, bonded or otherwise affixed to the underlying speculum support. In the preferred embodiment, at least one of the openings on the sheath is configured to hook under and/or over the duckbills or lips of the speculum. Hooks include extensions of the sheath that are designed to fit over the end of a duckbill speculum and around duckbill, so that at least a portion of the underside of the duckbill is covered with the hook. The hook conforms with the shape of the end and underside of the duckbill. Thus in this embodiment, the sheath is held in pace by the tension in the sheath when it is placed over the end of the speculum.

4. Additives

The sheath may have adhesive to the outer side of its surface to allow better tissue adhesion during use. Sheath may have adherent coating to allow retention of cells or microbes for cytopathology or microbiological testing.

Alternatively, the sheath optionally contains a hemostatic agent on the outer surface to prevent bleeding. Suitable hemostatic agents include fibrin, fibrinogen, thrombin, phopspholipids, Ca⁺² salts, aluminum sulfate, aluminum acetate, witch hazel, and diatomaceous earth. The hemostatic agent may be applied to the surface as a powder or as a coating, such as in a hydrogel.

The sheath may contain within the sheath or as adherent coating on the sheath other pharmacological or therapeutic agents such as anti-infectives, antibiotics, anti-mitotics, antiproliferatives, anti-inflammatories, or other agents.

Optionally, the sheath contains an indicator, dye or stain to mark an area of abnormal tissue.

B. Specula

“Speculum” or “specula” as generally used herein refers to an insertable device that, when placed in a lumen, is opened inside the lumen, thereby expanding the lumen. The device is formed of a supportive material, such as metals and plastics. The specula may be disposable or non-disposable devices.

Examples of types of specula that may be covered with the sheath include nasal specula, ear specula, anal specula, and vaginal specula. Optionally, the speculum contains a means for affixing the sheath to the speculum.

II. Method of Using the Sheath

The sheath is placed on any type or any size of speculum. To prevent contamination, the sheath is used one-time on one patient.

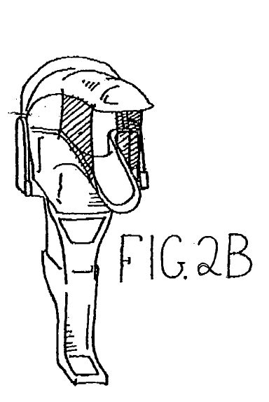

If the sheath contains a ringed end (10), the ringed end of the sheath is placed on the end of the speculum at the opening of the duckbill. As depicted in FIG. 2A, the sheath is then rolled onto the speculum until it covers the entire length of the duckbill speculum (15). When the sheath is properly applied to the speculum, the ring of latex will be at the end of the speculum (20) proximal to where the medical practitioner holds the speculum and controls the degree of separation and angular adjustment. The non-ringed end of the sheath will be at the distal end of the speculum, where the duckbill portion opens and closes (25). Alternatively, if the sheath contains two ringed openings or two openings without thicker rings of material, it is immaterial which opening is at which end of the speculum. Thus, the sheath is rolled onto the speculum, so that one opening is at the end proximal to where the medical practitioner holds the speculum (20) and one opening is at the end distal to where the medical practitioner holds the speculum (25).

If the sheath contains a hooked end, the sheath is placed on the end of the speculum at the opening of the duckbill in a rolled form with the hooked end rolled inside the sheath. As depicted in FIG. 2A, the sheath is then rolled onto the speculum until it covers the entire length of the duckbill speculum (15). When the sheath is properly applied to the speculum, the hooked end will be at the end of the speculum distal to where the medical practitioner holds the speculum and controls the degree of separation and angular adjustment and proximal to where the duckbill portion opens and closes (25). The hook is then affixed to at least one part of the duckbill in a manner that reduces or prevents slippage of the sheath upon insertion of the speculum into a patient's orifice.

If the sheath contains only one opening, the sheath is placed on the speculum so that the closed portion is located at the end of the speculum distal to where the medical practitioner holds the speculum (25) and the open portion is located at the end proximal to where the medical practitioner holds the speculum (20).

Once the speculum is inserted into the lumen and opened, the sheath stretches taut and holds back loose or redundant tissue and/or tissue that would otherwise fall into the space inside of the open specula. If the material is transparent, the sheath allows the medical practitioner to examine and view the lumen.

The disposable sheath for use with a speculum can be used with disposable or non-disposable specula. It can be easily removed from non-disposable speculums and then disposed of separately. A non-disposable speculum can be covered with a disposable sheath. This reduces or obviates the time spent cleaning and sterilizing the speculum. After examination or treatment, the speculum and sheath are removed from the patient and the sheath is discarded.

Alternatively, the sheath can cover a disposable speculum. In this embodiment, the sheath does not need to be removed from a disposable speculum prior to disposal of both pieces of equipment. In one embodiment, the sheath is removable from the speculum. In another embodiment, the sheath is permanently attached to a disposable vaginal speculum. For example, if a disposable vaginal speculum is being used, the speculum with the sheath on it is removed from the vagina and both the speculum and sheath are disposed of together. Optionally, the sheath is disposed of separately from the speculum.

III. Applications

The sheath is an inexpensive manner to increase the ease of examination and treatment of a lumen in a patient. For example, a transparent sheath allows for excellent visualization of vaginal tissues and of the cervix in women with loose vaginal tissue. Without use of a transparent sheath it would be difficult, if not impossible, to view the vagina and cervix well-enough to perform proper thorough assessments, evaluations and treatments.

The sheath is easily removed from the any type of speculum, if an area, such as the vagina or cervix, needs to be assessed, biopsied, or treated.

For vaginal applications, the sheath may facilitate procedures such as: routine gynecologic examination, examinations for infections and malignancies, pap smears, polypectomy, treatment of cervical abnormalities, treatment of human papilloma virus (HPV) in the vagina.

For nasal or otic examinations, the sheath may facilitate diagnostic examination and endoluminal therapies. It may facilitate submucous reduction of the nasal septum, polypectomy, sinus drainage or curettage procedures, eptorhinoplasty, microscopic or endoscopic sinus or otic surgeries, myringotomy, grommet insertion, or stapedectomy. For rectoanal purposes, the sheath may facilitate diagnostic and therapeutic procedures as well including external hemorrhoid repair. The sheath may be used in procedures for the repair of fistula-in ano, internal hemorrhoid repair, and cysto-recto-vaginal fistula repair.

The sheaths and methods for using the sheaths described above will be further understood with reference to the following non-limiting examples.

### EXAMPLES

### Example 1

### Sheath for Vaginal Speculum

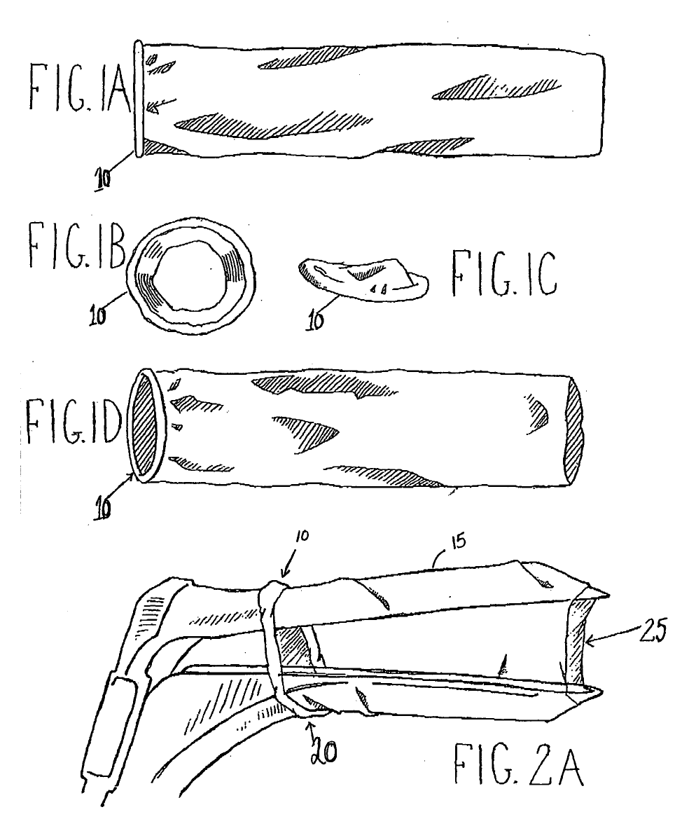

A sheath for a vaginal speculum is illustrated in FIGS. 1A, 1B, 1C, and 1D. As depicted in FIGS. 1A and 1D, the sheath may be cylindrical in shape and contain two openings. As depicted in FIGS. 1B and 1C, the sheath is in a rolled up form when it is placed onto the speculum.

The sheath in FIGS. 1A and 1D is approximately 10 cm long and the diameter of the opening at each end is roughly 3.5 cm. Its thickness is typically about 1 mm. The material from which the sheath is made can stretch when the vaginal speculum is opened to its maximum. A ring of thickened material (10) is present at only one end of the sheath. This ring is approximately 2 mm thick and is contiguous with the sheath at only one of the end openings.

Those skilled in the art will recognize, or be able to ascertain using no more than routine experimentation, many equivalents to the specific embodiments of the invention described herein. Such equivalents are intended to be encompassed by the following claims.

1. A sheath, wherein the sheath is formed of a compliant or partially compliant material, wherein the sheath comprises at least one opening, and wherein the sheath is configured to fit on a speculum.

2. The sheath of claim 1, wherein the material is selected from the group consisting of latex, vinyl, natural and synthetic rubbers, silicone, nylon, polyethylene, polypropylene, and non-degradable or degradable elastomers.

3. The sheath of claim 1, wherein the sheath comprises two openings.

4. The sheath of claim 1, wherein the sheath is configured to fit on a speculum selected from the group consisting of nasal specula, ear specula, anal specula, and vaginal specula.

5. The sheath of claim 4, wherein the sheath is configured to fit on a vaginal speculum.

6. The sheath of claim 1, wherein the sheath further comprises affixing means.

7. The sheath of claim 6, wherein the affixing means is selected from the group consisting of adhesives, hooks, ribs, buttons, and bonding materials.

8. The sheath of claim 1, wherein the sheath further comprises one or more active agents, selected from the group consisting of hemostatic agents, anti-infectives, antibiotics, anti-mitotics, antiproliferatives, and anti-inflammatories.

9. A kit comprising a sheath and a speculum, wherein the sheath is formed of a compliant or partially compliant material and wherein the sheath has at least one opening.

10. The kit of claim 9, wherein the material is selected from the group consisting of latex, vinyl, natural and synthetic rubbers, silicone, nylon, polyethylene, polypropylene, and non-degradable or degradable elastomers.

11. The kit of claim 9, wherein the sheath has two openings.

12. The kit of claim 9, wherein the speculum is selected from the group consisting of nasal specula, ear specula, anal specula, and vaginal specula.

13. The kit of claim 12, wherein the speculum is a vaginal speculum.

14. The kit of claim 9, wherein the sheath is affixed to the speculum.

15. The kit of claim 9, wherein the sheath further comprises one or more biologically active agents, selected from the group consisting of hemostatic agents, anti-infectives, antibiotics, anti-mitotics, antiproliferatives, and anti-inflammatories.

16. A method for examining or treating a lumen, comprising
inserting a device into the lumen, wherein the device comprises a sheath and a speculum, wherein the sheath is formed of a compliant or partially compliant material and wherein the sheath has at least one opening,
and opening the device in the lumen.

17. The method of claim 16, wherein the material is selected from the group consisting of latex, vinyl, natural and synthetic rubbers, silicone, nylon, polyethylene, polypropylene, and non-degradable or degradable elastomers.

18. The method of claim 16, wherein the sheath has two openings.

19. The method of claim 16, wherein the lumen is selected from the group consisting of nose, ear, rectum, and vagina.
